(19) United States
(12) Patent Application Publication
(10) Pub. No.: US 2025/0241524 A1
(43) Pub. Date: Jul. 31, 2025

(54) ENDOSCOPE WITH STEERING WIRE ASSEMBLY

(71) Applicant: AMBU A/S, Ballerup (DK)

(72) Inventor: Ismail KURU, Munich (DE)

(73) Assignee: AMBU A/S, Ballerup (DK)

(21) Appl. No.: 19/039,385

(22) Filed: Jan. 28, 2025

(30) Foreign Application Priority Data
Jan. 30, 2024 (DE) 10 2024 102 627.8

(51) Int. Cl. A61B 1/005; A61B 1/00; A61B 1/04

(52) U.S. Cl. CPC A61B 1/0052; A61B 1/00009; A61B 1/00043; A61B 1/0057; A61B 1/04

(57) ABSTRACT

Endoscope including a handle with a steering actuator, an insertion cord with a bending section, a first steering wire having a distal end connected to the bending section and a proximal end connected to the steering actuator, a second steering wire having a distal end connected to the bending section and a proximal end connected to the steering actuator, a ratchet rack arranged at a proximal end of at least one of the steering wires, and a rack connector arranged at the steering actuator.

### CROSS-REFERENCE TO RELATED APPLICATIONS

This application claims priority from and the benefit of German Patent Application No. 10 2024 102 627.8 filed Jan. 30, 2024; the disclosure of said application is incorporated by reference herein in its entirety.

### TECHNICAL FIELD

The present disclosure relates to endoscopes, and in particular endoscopes with a steering wire assembly, a system comprising an endoscope, and a method for assembly of endoscopes.

### BACKGROUND

Endoscopes are known and used for visual navigation into, and examination and diagnosis of, hollow organs and body cavities, as well as, optionally to assist in surgery, e.g. targeted tissue sampling. Endoscopes include procedure-specialized endoscopes, such as gastroscopes, duodenoscopes, ureteroscopes and bronchoscopes.

Single-use endoscopes optimize workflow and reduce cost while saving patient's lives and improving patient care. They optimize workflow and reduce cost because they are always ready when needed without the traditional large-scale capital and repair budgets required for reusable endoscopes. For example, a sterilization and storage facility is avoided, there is no need to maintain evidence of sterilization, and there is no need to transport endoscopes from sterilization and storage facilities to the buildings where they are needed, sometimes in the middle of the night or weekends. They save patient's lives and improve patient care because they are readily available and do not pose a cross-contamination risk. This also reduces hospital re-admissions. While single-use endoscopes are disposed after a single patient use (one or more procedures may be performed while the patient remains in the treatment room), the environmental impact of re-useable endoscopes, due to cleaning materials, CO₂ emissions during the cleaning process, and use of disposable personal protective equipment by personnel involved in transportation and sterilization of the re-useable endoscopes, can be similar to that of single-use endoscopes. To further reduce environmental impact, the endoscopes according to the present disclosure are primarily made of polymer materials.

To further enhance the benefits of single-use endoscopes, it is desirable to reduce manufacturing costs.

### SUMMARY

This disclosure relates to an endoscope comprising a steering wire, a steering actuator, a ratchet rack, a rack connector connected to the steering actuator, and a steering wire attached to the ratchet rack. The rachet rack enables assembly of the endoscope without tensioning the steering wire and tensioning of the steering wire after assembly by translating the rachet rack unidirectionally through the rack connector.

The possibility of assembling the endoscope without tensioning the steering wire allows for easy assembly with low variance and makes the construction suitable for automation, thereby reducing manufacturing costs. The ratchet rack and rack connector does not take up much space. The compactness means that the ergonomics of the endoscope may improve.

The endoscope may further comprise an insertion cord including a bending section. The endoscope may also comprise a first steering wire having a distal end connected to the bending section and a proximal end connected to the steering actuator. A second steering wire having a distal end connected to the bending section and a proximal end connected to the steering actuator may also be provided. A ratchet rack may be arranged at a proximal end of at least one of the steering wires. A rack connector may be arranged at the steering actuator, wherein the rack connector comprises a pawl.

Such an endoscope has several features. Provision of a ratchet rack and rack connector allows for simple assembly in that the steering wire may be connected to the steering actuator without being under tension. The steering wire may be tensioned at a later stage of assembly, right before shipment from a warehouse or even by the user at the moment of using the endoscope. The alternative where the steering wire is tensioned to the required tension, while keeping the endoscope in the correct position, and then permanently connected to the steering actuator, e.g. by gluing, while under tension, is more demanding. For some endoscopes, the steering wire is wound onto a wire actuator and through openings to increase friction and grip of the wire to the actuator and enable connection by cluing. However, the high friction in this system means that it may be difficult to achieve the correct tension in the steering wire of the endoscope. When the steering wire is tensioned in such a system it is tensioned to a certain pull force of the proximal end (free end) of the steering wire, but part of the pull force is taken up by the friction in the system, so the actual tension in the steering wire between the wire actuator and the bending section is unknown and may vary. Further, the steering wire must be kept steady during the curing process of the glue, which may be difficult and will often require complex machinery as it is difficult to perform this manually without large variation.

By decoupling connection and tensioning it is possible to achieve a more precise and correct tension of the steering wire, as the level of friction between steering wire and wire actuator is very low and will not influence the tensioning. The tension of the steering wire in this system will hence be equal to the pull force applied to the proximal end of the steering wire (i.e. ratchet rack).

The steering wire may be provided with the ratchet rack at any time before assembly, such as a sub-assembly.

Tensioning of the steering wire can be accomplished by pulling the free end of the ratchet rack. As such this is a linear movement, which is ergonomically beneficial compared to e.g. twisting, so the risk of injury due to repetitive work is minimum. Further pulling the free end of the ratchet rack can be automated simply, e.g. by pulling on an assembled endoscope.

Another aspect relates to a system comprising such an endoscope. The endoscope may further comprise an image device. The system may further comprise a video processing apparatus (VPA) capable of processing a video or image signal received from the endoscope, and a coupled display module outputting the video or image signal.

A further aspect relates to a method for assembling an endoscope. The method can comprise providing a handle, providing a steering actuator, providing a first steering wire, providing a second steering wire, providing a ratchet rack at a proximal end of at least one of the steering wires, and inserting the ratchet rack in a rack connector at the steering actuator. This method may simplify assembly, can be done with limited training and limited need for special tools or machinery, and may as such reduce the cost of assembly.

### BRIEF DESCRIPTION OF THE DRAWINGS

The disclosure will now be made in greater detail based on non-limiting embodiments and with reference to the schematic drawings on which:

FIG. 1 is a side view of an embodiment of an endoscope connectable to a monitor to form a visualization system,

FIG. 2 is a side view of the endoscope of FIG. 1 with parts removed,

FIG. 3 is a side view of a handle of another embodiment of an endoscope,

FIG. 4 shows the interior of the handle of FIG. 3 during assembly,

FIG. 5 shows an embodiment of a wire drum,

FIG. 6 shows the wire drum of FIG. 5 with steering wires mounted,

FIG. 7 is a close-up of an embodiment of a steering wire with a ratchet rack,

FIG. 8 is a schematic sectional view of the ratchet rack inserted in a connector,

FIG. 9 is an exploded side view of the wire drum of FIG. 5 and a control wheel,

FIG. 10 is a cross-sectional view taken along the line X-X of FIG. 9, and

FIG. 11 is a diagram illustrating an embodiment of assembling an endoscope.

### DETAILED DESCRIPTION

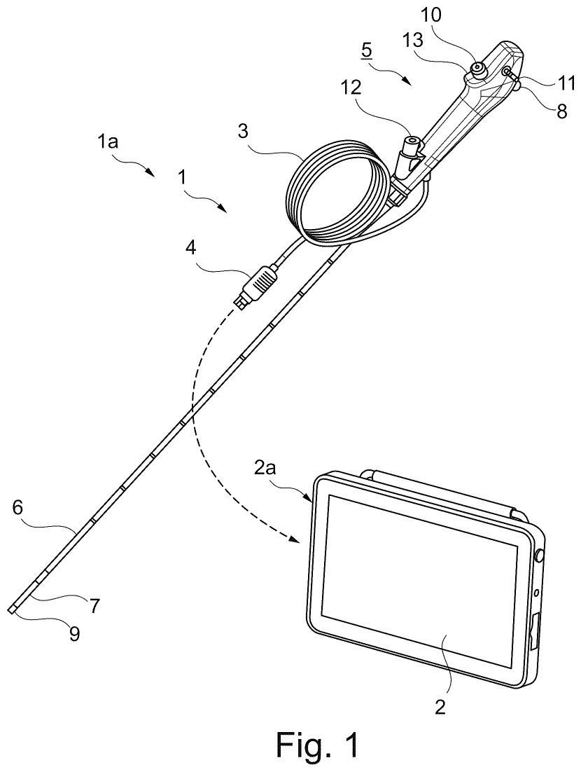

Turning first to FIG. 1, a visualization system 1a comprising an endoscope 1 and a VPA 2a including a display 2 is illustrated. The endoscope 1 may be connected to the VPA 2a via an umbilical cord 3 with a connector 4 insertable in a corresponding socket of the VPA 2a. Alternatively, there may be a wireless connection transmitting image data from the endoscope to the VPA, and potentially also transmitting control instructions from the VPA to the endoscope, e.g. controlling image capture or light. The endoscope comprises a handle 5 and an insertion cord 6 extending from the handle to the distal end of the endoscope.

The insertion cord 6 may include an insertion tube extending from the handle to a highly bendable, e.g. articulated, bending section 7, which is controllable by the operator via a steering actuator 8. The insertion cord may further include a distal tip arranged distally to the bending section, and the distal tip may comprise an observation optical system. The bending section 7 may be controllable by steering wires having a distal end connected at the bending section distal end and extending to the handle at the proximal end. The steering wire proximal end may be connected to a steering actuator 8 to tension or slack the steering wires, thereby bending the bending section 7. The steering wires may be Bowden cables comprising a steering wire arranged inside a wire pipe or sheath. More generally, the endoscope may comprise an interface at the proximal end connected to the insertion cord. The handle is an example of the interface. Another example is an interface configured for cooperation with drive means, e.g. for implementation of robotic endoscopy.

The illustrated endoscope is a two-way endoscope, meaning that the bending section can be bent in two directions, such as up and down. The bending section 7 is controllable by the steering actuator 8 at the handle, or interface, connected to the bending section 7 via steering wires running through the insertion cord, as will be discussed in more detail below. At the distal end of the bending section a distal tip 9 is provided. The distal tip 9 may comprise an imaging device, e.g. an image sensor, and illumination means, such as a light emitter. Example light emitters comprise a light emitting diode (LED) and a distal end of an optical fibre transmitting light from a light source. The light source may be in the handle 5, in the VPA, or connected to but external of the handle 5 and the VPA. There may further be provided a working channel (not shown) for tools.

The handle 5 may comprise a suction activation button 10 for activating suction from an external vacuum source (not shown) via a suction connector 11 through a suction channel having an opening at the distal end of the endoscope. The working channel may double as suction channel. The handle may also have a tool insertion port 12 for insertion of a tool through the handle and the working channel to an opening at the distal tip 9. Further the handle may comprise buttons 13 for activating various functions, such as controlling function of the image capture, such as taking of still images.

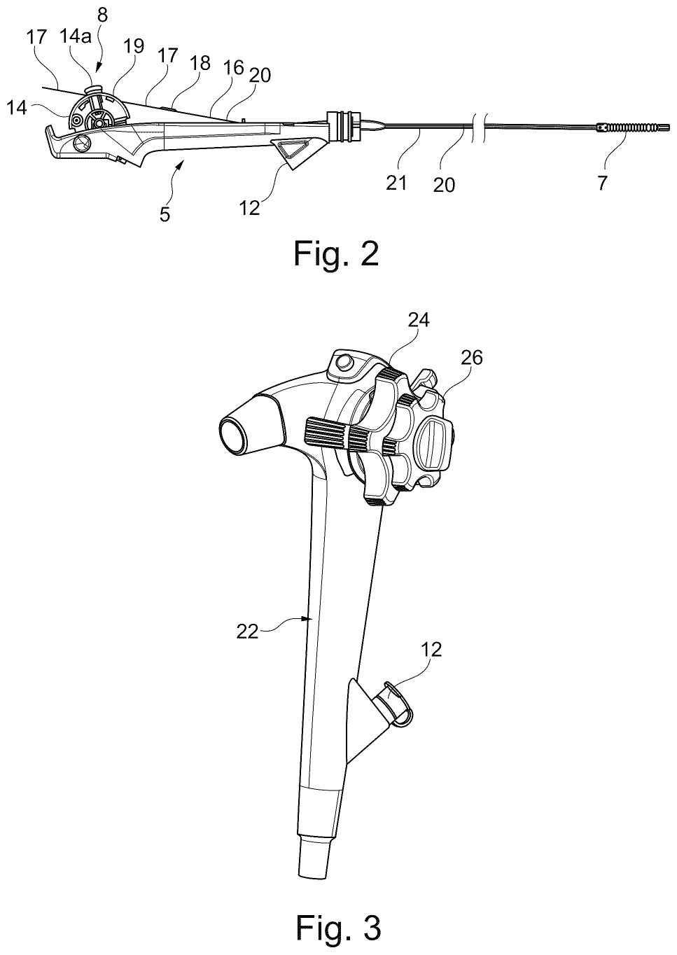

The interior of the handle 5 is shown in FIG. 2 with parts removed for illustration purposes. A handle cover part is removed and further an insertion tube and bending cover of the insertion cord are removed. In the present embodiment, the steering actuator comprises a wire roller 14 and a lever 14a mounted on the wire roller 14, which is rotatably mounted in the handle. A first steering wire 16 has a proximal end connected to the wire roller 14 and a distal end connected to the distal end of the bending section 7. As schematically illustrated, the first steering wire 16 may connect to a ratchet rack 17, e.g. by a crimp 18, and the ratchet rack 17 may in turn be inserted in a rack connector 19 at the wire roller 14 to connect thereto. The ratchet rack 17 has teeth and the connector has a detent mechanism as will be described further below. The ratchet rack 17 and connector 19 provide a one-way attachment in that the ratchet rack 17 can be advanced further into the rack connector 19, thereby tensioning the steering wire 16, whereas the teeth and detent mechanism of the ratchet rack and rack connector, respectively, engage and prevent retraction of the ratchet rack 17 from the rack connector 19. A second steering wire (not shown) is connected at a proximal end to the wire roller 14 and at a distal end to the distal end of the bending section 7.

The second steering wire may be connected to the wire roller 14 via a rack connector or directly to the wire roller 14 using any other suitable way of connecting, such as using glue, heat staking, ultrasonic welding, a knot or the like. The steering wires run inside wire pipes 20, 21 to thereby form Bowden cables. The wire pipes 20, 21 run from the handle to a position near the proximal end of the bending section 7. The wire pipes are normally kept stationary, whereas the steering wires are translatable inside the wire pipes to bend the bending section. The first steering wire 16 and the second steering wire may physically be one single wire body, which as soon as it has been threaded through and is suitably connected to the distal end of the bending section 7 cannot, from a pulling perspective, be distinguished from two individually fixed steering wires. In other words, even though there is conceptionally and functionally a first steering wire and a second steering wire, they may be constituted by one and the same physical wire and may be considered portions of one wire body, the first and second steering wires being connected to each other by a transition wire portion at the distal end of the bending section 5.

FIG. 3 illustrates a handle 22 of a four-way endoscope, i.e. an endoscope capable of bending the bending section in four directions, such as up, down, left and right. The illustrated endoscope handle comprises two steering actuators, each including a control wheel 24, 26 controlling bending in one set of directions, such as up-down or left-right. For simplicity other parts, such as umbilical cord and insertion cord are not shown. The control wheels 24, 26 are connected to wire drums which are also parts of the steering actuators of the present embodiment.

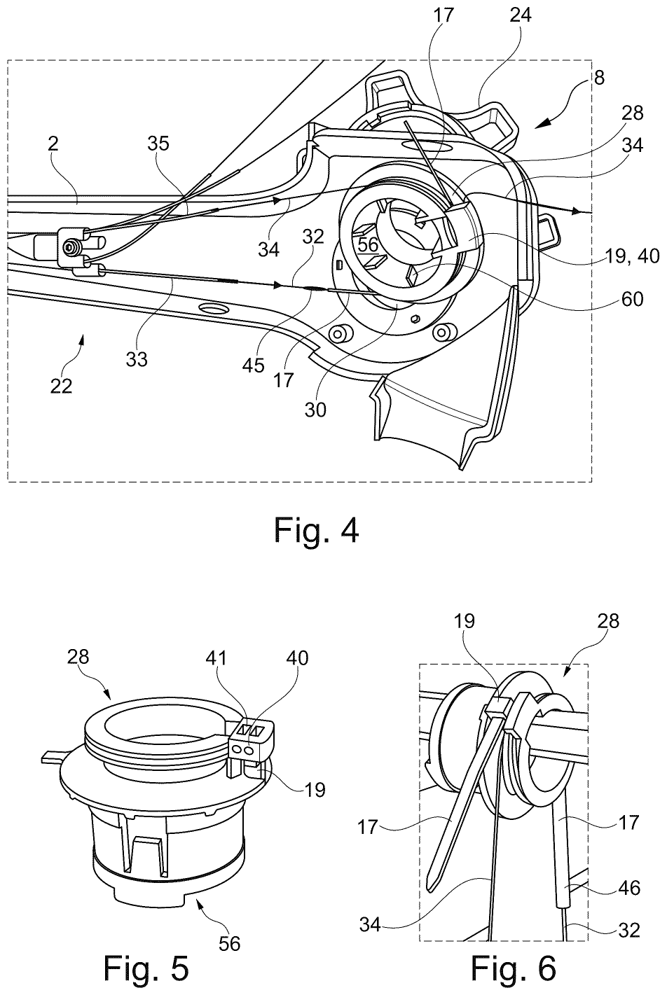

The inside of the handle 22 of FIG. 3 is seen in FIG. 4, which depicts a handle shell forming half a handle. A schematically illustrated, wire drum 28 is arranged inside the handle and connectable to the control wheel 24 outside the handle via a hole 30 in the handle shell, as will be discussed with reference to FIG. 8 below. The wire drum 28 is another part of the steering actuator 8 of the present embodiment. The first steering wire 32 extends from a wire pipe 33. The first steering wire 32 may connect to the ratchet rack 17 at a connection 45, as schematically illustrated. The ratchet rack 17 is inserted through the rack connector 19 on the wire drum 28. The second steering wire may be connected directly to the wire drum 28 by being threaded through wire connector 40 and pulled as illustrated by an arrow as schematically illustrated. This will be discussed in more detail with reference to FIG. 5 below. Alternatively, the second steering wire is connected indirectly to the wire drum 28 via a second ratchet rack 17 inserted through a second rack connector 19. A second set of steering wires 38, 39 are also illustrated in FIG. 5. The second set of steering wires connect to another wire drum (not shown) which in turn connect to the second control wheel (not shown). One or both of the second set of steering wires may comprise a ratchet rack, and the other wire drum may similarly comprise one or two rack connectors.

The wire drum 28 is shown in FIG. 5. The illustrated wire drum 28 comprises a wire connector 40 and a rack connector 19. The wire drum of FIG. 5 is illustrated, in FIG. 6, with steering wires 32, 34 attached. The first steering wire 32 is attached to the ratchet rack 17, as will be described in more detail below. The ratchet rack 17 may be inserted in the rack connector 19 of the wire drum 28 as illustrated. The rack connector 19 encircles the ratchet rack 17 inserted in the rack connector 19, and further the rack connector comprises a pawl engaging teeth of the ratchet rack preventing removal of the ratchet rack 17 from the rack connector 19 as will be described in more detail with reference to FIG. 8 below. The second steering wire 34 is wound around the wire drum 28 and threaded through the wire connector 40. The second steering wire 34 is permanently connected to the wire connector 40 with adhesive, which can be added through a glue port 41. The wire drum 28 may comprise two rack connectors if both steering wires comprise a ratchet rack.

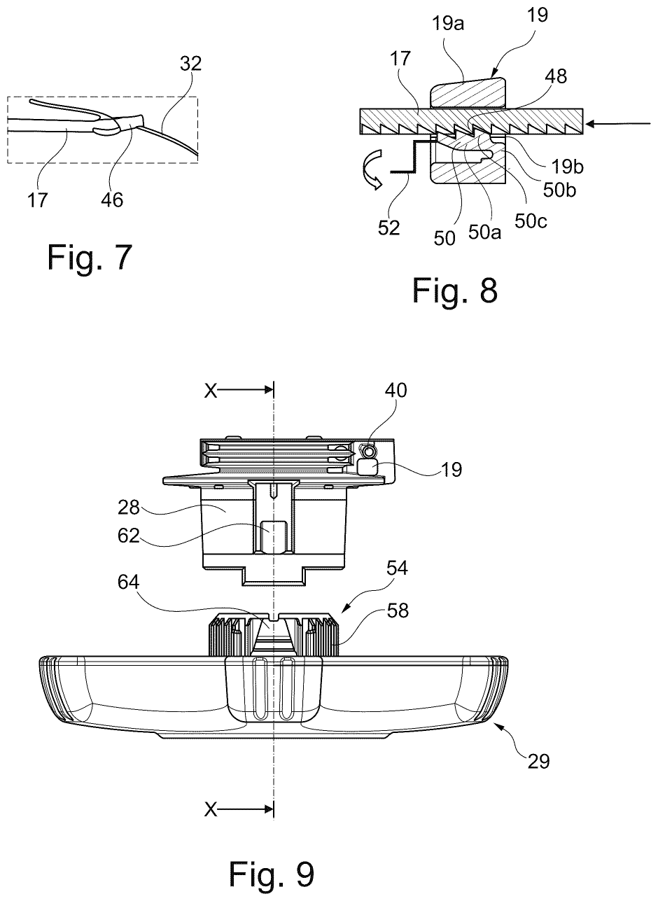

A detail of the first steering wire 32 and attachment to the ratchet rack 17 is shown in FIG. 7. The connection between wire and ratchet rack may be made by making a knot of the wire to the ratchet rack. The ratchet rack may comprise a lateral small slot in which the wire can positioned, with a knot on one side of the slot and adhesive applied onto the slot and the knot to secure the wire. Alternatively or additionally, glue 46 may be applied as illustrated for a more secure connection. Other alternatives for the connection include using a crimp, by heat staking or ultrasonic welding.

A cross-sectional view of an embodiment of the rack connector 19, with the ratchet rack 17 inserted, is illustrated in FIG. 8. Teeth 48 of the ratchet rack 17 engage the pawl 50. The pawl 50 is flexible or at least flexibly hinged, so that travel of the ratchet rack 17 is possible in one direction (unidirectional), but not in the opposite direction. The rack connector 19 comprises a body 19a having a cavity 19b. The pawl 50 comprises an arm 50a which comprises teeth 50c. A hinge 50b connects the arm to the body 19a. As shown, the hinge is comprised of a narrow amount of polymer material, which is resiliently flexible. In one direction, the pawl 50 flexes (the hinge 50b is flexed by the lever) as the teeth 50a ride on the inclined teeth 48 of the ratchet rack, as schematically indicated by an arrow. In the other direction, the pawl 50 engages the teeth 48 of the ratchet rack and the ratchet rack is hence restricted from being disconnected. The width of the cavity 19b is sufficiently small to prevent disengagement of the ratchet rack 17, thereby the ratchet rack 17 can be connected to the rack connector 19 without a risk of disconnection of the ratchet rack. The rack connector 19 may be injection molded and then affixed to the wire drum 28 or, preferably, it may be molded together with the wire drum 28 in a one-piece part.

The ratchet and pawl connection make it possible to connect the steering wire to the wire drum first by inserting the ratchet rack 17 in the rack connector 19 and at a later stage tension the steering wire by advancing the ratchet rack in the rack connector 19. In the illustration, the pawl comprises three teeth for engagement with the ratchet rack 17, which will ensure a secure connection between rack and pawl and hinder or at least counteract risk of disengagement or failure of the engagement. In the simplest form the pawl may be an inclined arm adapted to give way in one direction but engage a tooth of the ratchet rack in the other direction. On one side the teeth slope at an angle, whereas on the other side the teeth are near perpendicular to the longitudinal direction of the ratchet rack. This is a unidirectional construction is that when advancing the ratchet rack in one direction the pawl of the rack connector rides on the slope side of the teeth, whereas an attempt to move the ratchet rack in the other direction would stop as the pawl engage a near perpendicular side of the teeth.

A more robust connection can be achieved by increasing the number of pawl engagement points. Further the size of the teeth may be chosen to have a more, or less, robust connection. The width of the ratchet rack 17 may also be chosen depending on the situation. In some cases, the focus is to provide a very compact design to reduce the size of the construction and possibly also the amount of material used, in which case the ratchet rack 17 is narrow and the teeth 48 are small. In other cases, the focus is on providing a robust connection, in which the ratchet rack 17 is wide, and the teeth 48 are large. This may be the case where the insertion cord is long. The length and size of the insertion cord may require different amount of bending force to bend the bending section of the endoscope, e.g. due to inevitable friction in the system. Further the use of the endoscope may require different amounts of bending force, e.g. whether the endoscope is a type used in a long, twisted channel, like colon, which requires a high bending force, or a type used in a cavity, like the bladder, which requires a relatively low bending force. The ratchet rack may have teeth on more than one side interacting with corresponding teeth arranged on the rack connector.

The illustrated variant of the rack connector 19 comprises an optional pawl release 52, which may be pressed as indicated by the arrow to disengage the pawl 50 from the teeth 48 of the ratchet rack 17. Hereby the rack connector 19 may be partially or fully retracted from the rack connector 19. This may be beneficial if the steering wire is accidentally tensioned too much. Further a release mechanism may facilitate disassembly thereby making reuse of different parts or recycling of materials easier hereby lowering cost and the carbon footprint of the product.

The other way round is also possible by having a ratchet rack with flexible teeth engaging one or more recesses in the rack connector. This is generally a less strong connection but may suffice for some types of endoscopes.

It is also possible to attach the rack connector to the steering wire and have the ratchet connected to the steering actuator.

Other constructions providing unidirectional translation are also possible, such as cam cleats used to pinch and hold a string connected to the steering wire or to pinch the steering wire directly.

A general feature is that the ratchet rack and rack connector do not take up much space. This may be relevant in some cases as space can be a limiting factor in the handle of an endoscope. The exterior size of the handle is limited by ergonomics, especially for users having relatively small hands. The interior space of the handle available for steering wires and control is limited by the different features that occupy the space, such as suction channel and suction valve.

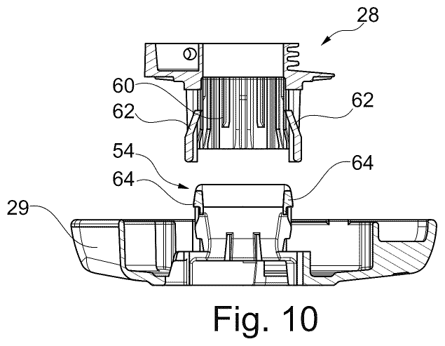

A combination of the wire drum 28 and the control wheel 29 can be seen in FIGS. 9 and 10. The wire drum 28 and control wheel 29 are here shown aligned for connection. An engagement part 54 of the control wheel 29 is adapted for insertion in the hollow interior 56 of the wire drum (shown in FIG. 4). The outer periphery of the engagement part 54 may comprise splines 58 for non-rotational engagement with complimentary splines 60 on the inner surface of the hollow interior 56 of the wire drum. Further the wire drum 28 may comprise a barb 62 adapted for engagement with a complimentary step 64 on the control wheel engagement part for securing the control wheel to the wire drum upon introduction of the control wheel engagement part 54 into the wire drum interior 56. Such a snap fit connection has the benefit that it makes the assembly process easy and quick. Further the snap fit connection is non-permanent and may be released at will, e.g. for disassembly at end of life of the endoscope to facilitate reuse of parts or recycling of materials. An alternative way of connecting is by gluing, which may have the advantage that the parts can be simpler and hence easier and cheaper to produce, but e.g. recycling can be more difficult, and further not all materials facilitate gluing.

Materials for the ratchet rack and the rack connector may be any suitable material, such as a polymer material. The steering wire may be a single, solid thread or made up of a number of filaments, e.g. braided. The steering wire may be made of a polymer material or metal. Polymer materials generally have lower cost and carbon footprint than metals, but on the other hand metals are generally stronger, which may be advantageous for high loads.

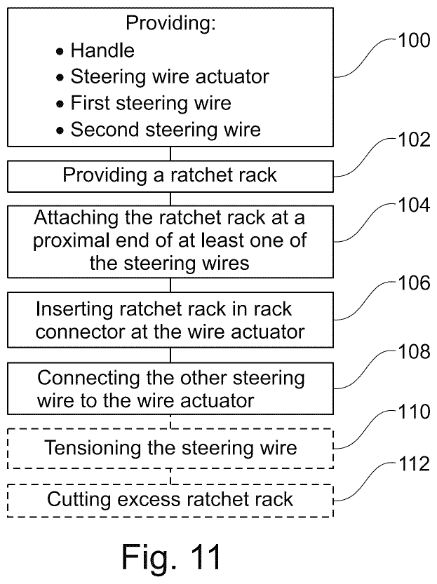

FIG. 11 schematically illustrates a method for assembly of an endoscope indicated by the flowchart 99. The method comprises, at 100, providing a handle, providing a steering actuator, providing a first steering wire, and providing a second steering wire. Further, the method comprises, at 102, providing a ratchet rack, and, at 104, attaching the ratchet rack at a proximal end of at least one of the steering wires, and, at 106, inserting the ratchet rack in a rack connector at the steering actuator. Further, the method comprises, at 108, connecting the other steering wire to the wire actuator. The method may further comprise, at 110, tensioning the steering wire. The method may further comprise, at 112, cutting excess ratchet rack.

With the method described in FIG. 11, the steering wire may be connected to the wire actuator without the steering wire being tensioned. This simplifies the assembly process in that connection and tensioning need not be performed at the same time and in the same apparatus, which could otherwise be a bottleneck in the assembly. Further it may be easier to control the quality of tensioning and connection separately rather than controlling both processes at the same time. With this method the tensioning may take place in a closed environment, such as inside the assembled handle, whereby some safety measures may be lifted. As such it is easier to keep an operator safe during assembly with this method.

The method for assembly may optionally include tensioning of the steering wire. This can be accomplished by pulling the free end of the ratchet rack. As such this as a linear movement, which is ergonomically beneficial compared to e.g. twisting, so the risk of injury due to repetitive work is minimum. Further it is considered relatively straightforward to automate such a process.

The method may optionally further include cutting excess ratchet rack.

The endoscope and method of assembly of the endoscope may be beneficial for both reusable and single-use endoscopes.

The following items are further variations and examples of the embodiments described with reference to the figures.
1. An endoscope comprising: a handle with a steering actuator; an insertion cord including a bending section; a first steering wire having a distal end connected to the bending section and a proximal end; a ratchet rack connected to the proximal end of the first steering wire; and a rack connector arranged at the steering actuator, wherein the rack connector comprises a pawl.
2. The endoscope of item 1, wherein the rack connector comprises a pawl release.
3. The endoscope of item 1 or 2, further comprising: a second steering wire having a distal end connected to the bending section and a proximal end; a second ratchet rack connected to the proximal end of the second steering wire; and a second rack connector arranged at the steering actuator, wherein the rack connector comprises a pawl.
4. The endoscope of any one of the items above, further comprising a second steering actuator, a third ratchet rack, a third rack connector attached to the second steering actuator, and a third steering wire, the third steering wire having a distal end connected to the bending section and a proximal end attached to the third ratchet rack, the third ratchet rack being inserted in the third rack connector.
5. The endoscope of item 4, further comprising a fourth steering wire having a distal end connected to the bending section and a proximal end attached to a ratchet rack inserted in a rack connector of the second steering actuator.
6. A system comprising an endoscope according to any one of the items above, wherein the endoscope further comprises an image device, the system further comprising a video processing apparatus capable of processing a video or image signal received from the endoscope, and a coupled display video outputting the video or image signal.
7. A method for assembling an endoscope, comprising providing a handle,
providing a steering actuator, providing a first steering wire, providing a second steering wire, providing a ratchet rack at a proximal end of at least one of the steering wires, inserting the ratchet rack in a rack connector at the steering actuator, and connecting the other steering wire to the steering actuator.
8. The method of item 7, further comprising tensioning the steering wire by pulling the ratchet rack.
9. The method of item 8, further comprising cutting excess ratchet rack at the rack connector.
10. Endoscope (1) comprising: a handle (5, 22) or interface with a steering actuator; an insertion cord (6) including a bending section (7); a first steering wire having a distal end connected to the bending section and a proximal end connected to the steering actuator; and means for unidirectional tensioning of the first steering wire.
12. Endoscope according to item 11, wherein the means for unidirectional tensioning is configured for application of a force directed in translational direction.

### LIST OF REFERENCE SIGNS

1 endoscope
2 display
3 umbilical cord
4 electrical connector
5 handle
6 insertion cord
7 bending section
8 lever
9 distal tip
10 suction activation button
11 suction connector
12 tool insertion port
13 buttons
14 wire roller
16 first steering wire
17 ratchet rack
18 crimp
19 rack connector
20, 21 wire pipe
22 handle
24, 26 control wheel
28 wire drum
30 hole
32, 34 steering wires
33,35 wire pipes
38,39 steering wires
40 wire connector
41 glue port
45 connection
46 glue
48 teeth
50 pawl
52 pawl release
54 engagement part
56 interior
58, 60 splines
62 barb
64 step
99 flowchart

1. An endoscope comprising:
a handle;
a steering actuator supported by the handle;
an insertion cord extending from the handle and including a bending section;
a first steering wire having a distal end and a proximal end, the distal end being connected to the bending section;
a first rack connector arranged at the steering actuator; and
a first ratchet rack connected to the proximal end of the first steering wire and to the first rack connector, the first ratchet rack being unidirectionally translatable in the first rack connector to tension the first steering wire.

2. The endoscope of claim 1, wherein the first rack connector comprises a pawl including teeth configured to engage teeth of the first ratchet rack to effect unidirectionally translation of the first ratchet rack in the first rack connector.

3. The endoscope of claim 1, wherein the first rack connector comprises a pawl and a pawl release.

4. The endoscope of claim 1, further comprising:
a second steering actuator supported by the handle;
a second steering wire having a distal end and a proximal end, the distal end being connected to the bending section;
a third steering wire having a distal end and a proximal end, the distal end of the third steering wire being connected to the bending section;
a fourth steering wire having a distal end and a proximal end, the distal end of the fourth steering wire being connected to the bending section;
a second rack connector arranged at the steering actuator;
a third rack connector arranged at the second steering actuator;
a fourth rack connector arranged at the second steering actuator;
a second ratchet rack connected to the proximal end of the second steering wire and to the second rack connector, the second ratchet rack being unidirectionally translatable in the second rack connector to tension the second steering wire;
a third ratchet rack connected to the proximal end of the third steering wire and to the third rack connector, the third ratchet rack being unidirectionally translatable in the third rack connector to tension the third steering wire; and
a fourth ratchet rack connected to the proximal end of the fourth steering wire and to the fourth rack connector, the fourth ratchet rack being unidirectionally translatable in the fourth rack connector to tension the fourth steering wire.

5. The endoscope of claim 1, the endoscope further comprising a second steering wire having a distal end and a proximal end, the distal end being connected to the bending section and the proximal end connected, directly or indirectly, to the first steering actuator.

6. The endoscope of claim 5, the endoscope further comprising:
a second steering actuator supported by the handle;
a third steering wire having a distal end and a proximal end, the distal end of the third steering wire being connected to the bending section;
a third rack connector arranged at the second steering actuator; and
a third ratchet rack connected to the proximal end of the third steering wire and to the third rack connector, the third ratchet rack being unidirectionally translatable in the third rack connector to tension the third steering wire.

7. The endoscope of claim 1, further comprising:
a second steering wire having a distal end and a proximal end, the distal end being connected to the bending section;
a second rack connector arranged at the steering actuator; and
a second ratchet rack connected to the proximal end of the second steering wire and to the second rack connector, the second ratchet rack being unidirectionally translatable in the second rack connector to tension the second steering wire.

8. The endoscope of claim 7, wherein the second rack connector comprises a pawl and a pawl release.

9. The endoscope of claim 7, the endoscope further comprising:
a second steering actuator supported by the handle;
a third steering wire having a distal end and a proximal end, the distal end of the third steering wire being connected to the bending section;
a third rack connector arranged at the second steering actuator; and
a third ratchet rack connected to the proximal end of the third steering wire and to the third rack connector, the third ratchet rack being unidirectionally translatable in the third rack connector to tension the third steering wire.

10. The endoscope of claim 9, wherein the third ratchet rack is inserted in the third rack connector.

11. The endoscope of claim 9, wherein the third rack connector comprises a pawl.

12. The endoscope of claim 9, the endoscope further comprising:
a fourth steering wire having a distal end and a proximal end, the distal end of the fourth steering wire being connected to the bending section;
a fourth rack connector arranged at the second steering actuator; and
a fourth ratchet rack connected to the proximal end of the fourth steering wire and to the fourth rack connector, the fourth ratchet rack being unidirectionally translatable in the fourth rack connector to tension the fourth steering wire.

13. The endoscope of claim 9, further comprising a fourth steering wire having a distal end connected to the bending section and a proximal end attached to the second steering actuator.

14. The endoscope of claim 1, the endoscope further comprising:
a second steering wire having a distal end and a proximal end, the distal end of the second steering wire being connected to the bending section;
a second steering actuator supported by the handle;
a third steering wire having a distal end and a proximal end, the distal end of the third steering wire being connected to the bending section;
a fourth steering wire having a distal end and a proximal end, the distal end of the fourth steering wire being connected to the bending section;
a second rack connector arranged at the second steering actuator; and
a second ratchet rack connected to the proximal end of the third steering wire and to the second rack connector, the second ratchet rack being unidirectionally translatable in the second rack connector to tension the third steering wire.

15. A system comprising:
the endoscope of claim 1;
an imaging sensor; and
a video processing apparatus configured to receive a video or image signal from the endoscope.

16. The system of claim 15, wherein the video processing apparatus comprises a display and is configured to present video or images with the display, the video or images being based on the video or image signals from the endoscope.

17. The system of claim 15, wherein the endoscope further comprises:
a second steering actuator supported by the handle;
a second steering wire having a distal end and a proximal end, the distal end being connected to the bending section;
a third steering wire having a distal end and a proximal end, the distal end of the third steering wire being connected to the bending section;
a fourth steering wire having a distal end and a proximal end, the distal end of the fourth steering wire being connected to the bending section;
a second rack connector arranged at the second steering actuator; and
a second ratchet rack connected to the proximal end of the third steering wire and to the third rack connector, the third ratchet rack being unidirectionally translatable in the third rack connector to tension the third steering wire.

18. The system of claim 15, wherein the first rack connector comprises a pawl including teeth configured to engage teeth of the first ratchet rack to effect unidirectionally translation of the first ratchet rack in the first rack connector.

19. A method of assembling the endoscope of claim 1, the method comprising:
providing the handle;
providing the first steering actuator;
providing the first steering wire;
providing the ratchet rack;
providing the rack connector;
inserting the ratchet rack in the rack connector at the steering actuator; and
connecting the proximal end of the first steering wire to the steering actuator.

20. The method of claim 19, further comprising tensioning the first steering wire by pulling on the ratchet rack.
